(19) United States
(12) Patent Application Publication
(10) Pub. No.: US 2021/0259617 A1
(43) Pub. Date: Aug. 26, 2021

(54) PHYSIOLOGICAL INFORMATION PROCESSING APPARATUS, PHYSIOLOGICAL INFORMATION PROCESSING METHDO AND STORAGE MEDIUM

(71) Applicant: NIHON KOHDEN CORPORATION, Tokyo (JP)

(72) Inventors: Masami TANISHIMA, Tokorozawa-shi (JP); Masumi KUBOTA, Tokorozawa-shi (JP); Takashi MATO, Tokyo (JP)

(21) Appl. No.: 17/176,390

(22) Filed: Feb. 16, 2021

(30) Foreign Application Priority Data
Feb. 25, 2020 (JP) 2020-029671

(51) Int. Cl. A61B 5/388; A61B 5/00; G16H 10/60; G16H 50/30; G16H 50/70; G16H 15/00

(52) U.S. Cl. CPC A61B 5/388; A61B 5/743; A61B 5/4035; A61B 5/72; A61B 5/318; G16H 50/30; G16H 50/70; G16H 15/00; G16H 10/60

(57) ABSTRACT

A physiological information processing apparatus including: a processor; and a memory that stores a computer-readable command. When the computer-readable command is executed by the processor, the physiological information processing apparatus acquires physiological information data indicating physiological information of a subject, acquires a value of a parameter relevant to an autonomic nerve function of the subject in each of first time intervals based on the physiological information data, acquires an abnormality index value indicating an extent of abnormality in the autonomic nerve function of the subject based on the values of the parameter acquired in the first time intervals respectively, and displays information relevant to the abnormality index value.

### CROSS-REFERENCE TO RELATED APPLICATIONS

The present application claims priority from Japanese Patent Application No. 2020-029671, filed Feb. 25, 2020, the entire content of which is incorporated herein by reference.

### TECHNICAL FIELD

The present disclosure relates to a physiological information processing apparatus and a physiological information processing method. Further, the present disclosure relates to a program for making a computer execute the physiological information processing method, and a computer-readable storage medium in which the program is stored.

### BACKGROUND ART

There has been disclosed an apparatus for monitoring activity of a patient relevant to his/her autonomic nerve (e.g. see JP2005-261777A). JP2005-261777A discloses an autonomic nerve activity monitoring apparatus that can estimate or determine abnormal reaction of a living body of a patient based on his/her autonomic nerve activity. In order to make the autonomic nerve activity of the patient visible, frequency analysis is performed on heart rate variability (HRV) of the patient by the autonomic nerve activity monitoring apparatus so that a trend graph indicating a change of a high frequency component (HF) of the heart rate variability over time and a trend graph indicating a change of a ratio (LF/HF) of a low frequency component (LF) to the high frequency component over time can be displayed on a display screen of the autonomic nerve activity monitor apparatus.

Now, a trend graph which indicates a change of the autonomic nerve function (consisting of a sympathetic nerve function and a parasympathetic nerve function) of the patient over time is displayed on the monitor apparatus. A medical worker who carefully observes the trend graph may notice abnormality in the autonomic nerve function of the patient. However, there is a possibility that the medical worker cannot accurately grasp an extent of a disease of the patient relevant to the abnormality in the autonomic nerve function. Particularly if the medical worker does not have enough experience, it can be supposed that the medical worker has difficulty in accurately grasping the extent of the disease of the patient relevant to the abnormality in the autonomic nerve function of the patient by only visibly recognizing the trend graph indicating the change of the autonomic nerve function over time. Thus, there is room for an improvement in user-friendliness of such a physiological information processing apparatus from the aforementioned viewpoint.

### SUMMARY

An object of the present disclosure is to provide a physiological information processing apparatus and a physiological information processing method which can contribute to diagnosis of a subject's disease relevant to abnormality in his/her autonomic nerve function. In addition, another object of the present disclosure is to provide a program for making a computer execute the physiological information processing method, and a computer-readable storage medium in which the program is stored.

A physiological information processing apparatus according to an aspect of the present disclosure includes:

a processor; and

a memory that stores a computer-readable command.

When the computer-readable command is executed by the processor, the physiological information processing apparatus:

acquires physiological information data indicating physiological information of a subject;

acquires a value of a parameter relevant to an autonomic nerve function of the subject in each of first time intervals based on the physiological information data;

acquires an abnormality index value indicating an extent of abnormality in the autonomic nerve function of the subject based on the values of the parameter acquired in the first time intervals respectively; and

displays information relevant to the abnormality index value.

A physiological information processing method according to another aspect of the present disclosure includes:

a step of acquiring physiological information data indicating physiological information of a subject;

a step of acquiring a value of a parameter relevant to an autonomic nerve function of the subject in each of first time intervals based on the physiological information data;

a step of acquiring an abnormality index value indicating abnormality in the autonomic nerve function of the subject based on the values of the parameter acquired in the first time intervals respectively, and

a step of displaying information relevant to the abnormality index value.

A program for making a computer execute the physiological information processing method and a computer-readable storage medium in which the program is stored are provided.

According to the present disclosure, it is possible to provide a physiological information processing apparatus and a physiological information processing method that can contribute to diagnosis of a subject's disease relevant to abnormality in his/her autonomic nerve function.

### BRIEF DESCRIPTION OF DRAWINGS

FIG. 1 is a hardware configuration diagram showing a physiological information processing apparatus according to an embodiment of the present disclosure (hereinafter referred to as present embodiment).

FIG. 2 is a flowchart for explaining a physiological information processing method according to the present embodiment.

FIG. 3 is a flowchart showing an example of a process of acquiring values of parameters relevant to an autonomic nerve function of a patient.

FIG. 4 is a view showing an example of a trend graph indicating a change of an HF parameter over time and a trend graph indicating a change of an LF/HF parameter over time.

FIG. 5 is a flowchart showing an example of a process of acquiring abnormality index values.

FIG. 6 is a view for explaining the abnormality index values.

FIG. 7 is a view showing the trend graph of the HF parameter and the trend graph of the LF/HF parameter which are displayed on a display screen of a display, and an example of message information determined based on the abnormality index values.

FIG. 8A shows a trend graph of a variation width of the HF parameter, a trend graph of a variation width of the LF/HF parameter, a trend graph of a number of times of abnormal enhancement of the HF parameter, and a trend graph of a number of times of abnormal enhancement of the LF/HF parameter, and FIG. 5B shows a trend graph of a reference value of the HF parameter and a trend graph of a reference value of the LF/HF parameter.

FIG. 9 is a view showing an example of a radar chart showing values of the abnormality index values.

### DESCRIPTION OF EMBODIMENT

The present embodiment will be described below with reference to the drawings. Description about elements having the same reference signs as elements that have been already described in description of the present embodiment will be omitted for convenience of explanation.

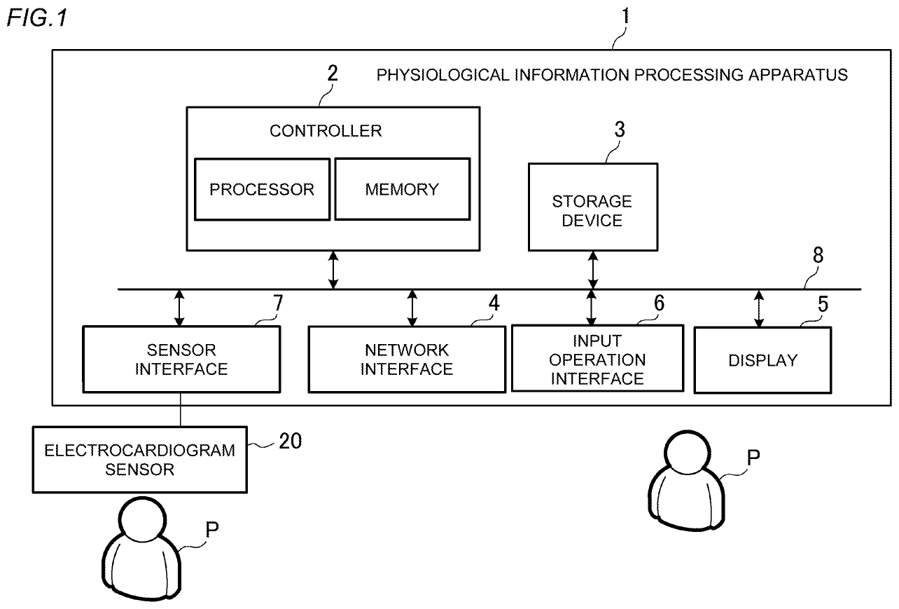

FIG. 1 shows a hardware configuration diagram of a physiological information processing apparatus 1 according to the present embodiment. As shown in FIG. 1, the physiological information processing apparatus 1 (hereinafter simply referred to as processing apparatus 1) is provided with a controller 2, a storage device 3, a network interface 4, a display 5, an input operation interface 6, and a sensor interface 7, which are communicably connected to one another via a bus 8.

The processing apparatus 1 may be a dedicated apparatus (such as a physiological information monitor) for displaying a trend graph of vital signs of a patient P (subject). For example, the processing apparatus 1 may be a personal computer, a workstation, a smartphone, a tablet, or a wearable device (such as a smartwatch or AR glasses) worn on a body (e.g. an arm, the head, or the like) of an operator U (medical worker).

The controller 2 is provided with a memory and a processor. The memory is configured so as to store computer-readable commands (programs). For example, the memory may be constituted by a Read Only Memory (ROM) where various programs etc. have been stored, a Random Access Memory (RAM) which has work areas where various programs etc. executed by the processor can be stored, etc. In addition, the memory may be constituted by a flash memory etc. The processor is, for example, constituted by a Central Processing Unit (CPU), a Micro Processing Unit (MPU) and/or a Graphics Processing Unit (GPU). The CPU may be composed of a plurality of CPU cores. The GPU may be composed of a plurality of GPU cores. The processor may be configured so as to expand, onto the RAM, a program designated from the various programs incorporated into the storage device 3 or the ROM, and execute various processes in cooperation with the RAM.

The controller 2 may control various operations of the processing apparatus 1 particularly by using the processor to expand a physiological information processing program which will be described later onto the RAM, and execute the program in cooperation with the RAM. Details of the physiological information processing program will be described later.

The storage device 3 which is, for example, a storage device (storage) such as a Hard Disk Drive (HDD), a Solid State Drive (SSD), or a flash memory is configured to store programs or various data. The physiological information processing program may be incorporated into the storage device 3. In addition, physiological information data (particularly electrocardiogram data) indicating physiological information of the patient P (subject) may be stored in the storage device 3. For example, electrocardiogram data acquired by an electrocardiogram sensor 20 may be stored in the storage device 3 via the sensor interface 7.

The network interface 4 is configured so as to connect the processing apparatus 1 to a communication network. Specifically, the network interface 4 may include various wired connection terminals for making communication with an external apparatus such as a server through the communication network. Furthermore, the network interface 4 may include an RF circuit and an antenna for making wireless communication with the external apparatus. The wireless communication standard between the external apparatus and the processing apparatus 1 is Wi-Fi (registered trademark), Bluetooth (registered trademark), ZigBee (registered trademark) or LPWA. The communication network is a Local Area Network (LAN), a Wide Area Network (WAN), the Internet, or the like. For example, the physiological information processing program or the physiological information data may be acquired from the server disposed on the communication network through the network interface 4.

The display 5 may be a display device such as a liquid crystal display or an organic EL display, or may be a display device such as a transmissive type or non-transmissive type head mounted display to be worn on the head of the operator. Further, the display 5 may be a projector for projecting an image onto a screen. The processing apparatus 1 may be not provided with the display 5. For example, output data outputted from the processing apparatus 1 may be displayed on a display device of an external apparatus such as a central monitor through the network interface 4 or an input interface (not shown).

The input operation interface 6 is configured so as to accept an input operation from the operator U (medical worker) operating the processing apparatus 1, and generate an instruction signal corresponding to the input operation. The input operation interface 6 is, for example, a touch panel disposed to be superimposed on the display 5, operation buttons provided in a housing of the processing apparatus 1, a mouse and/or a keyboard, or the like. After the instruction signal generated by the input operation interface 6 has been transmitted to the controller 2 through the bus 8, the controller 2 executes a predetermined operation in accordance with the instruction signal.

The sensor interface 7 is an interface for connecting the electrocardiogram sensor 20 to the processing apparatus 1. The electrocardiogram sensor 20 has, for example, a plurality of electrodes (e.g. three electrodes) attached to the chest of the patient P. The electrocardiogram sensor 20 is configured so as to make contact with a measurement region of the patient P and detect a change of potential in the measurement region. The sensor interface 7 may be physically connected to a cable connector of the electrocardiogram sensor 20. The sensor interface 7 may have an analog processing circuit including a differential amplifier and an AD converter. The differential amplifier is configured so as to amplify a difference between a potential outputted from one measurement electrode of the electrocardiogram sensor 20 and a potential outputted from the other measurement electrode of the electrocardiogram sensor 20 to thereby generate electrocardiogram data (analog data). The A/D converter is configured so as to convert the electrocardiogram data (analog data) into digital data. After the electrocardiogram data (digital data) have been generated thus by the sensor interface 7, the generated electrocardiogram data are stored in the memory or the storage device 3.

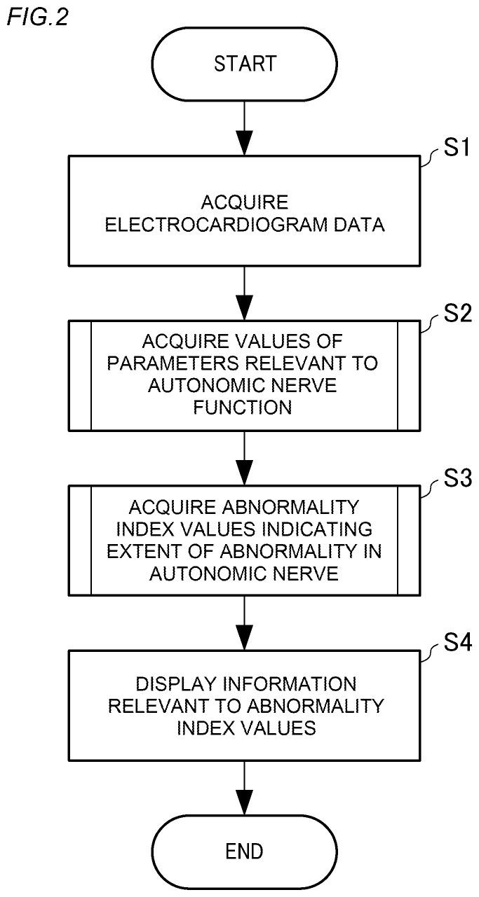

Next, a physiological information processing method according to the present embodiment will be described below with reference to FIG. 2. FIG. 2 is a flowchart for explaining the physiological information processing method according to the present embodiment.

As shown in FIG. 2, the controller 2 acquires, as physiological information data, electrocardiogram data indicating an electrocardiogram of a patient P in a step S1. Specifically, to acquire the physiological information data in real time, the controller 2 acquires the electrocardiogram data in real time through the electrocardiogram sensor 20 and the sensor interface 7. On the other hand, the controller 2 may acquire the electrocardiogram data stored in the storage device 3. The electrocardiogram data stored in the storage device 3 may be, for example, the electrocardiogram data of the patient P for 24 hours. Further, the controller 2 may acquire the electrocardiogram data through a server or the like disposed on the communication network.

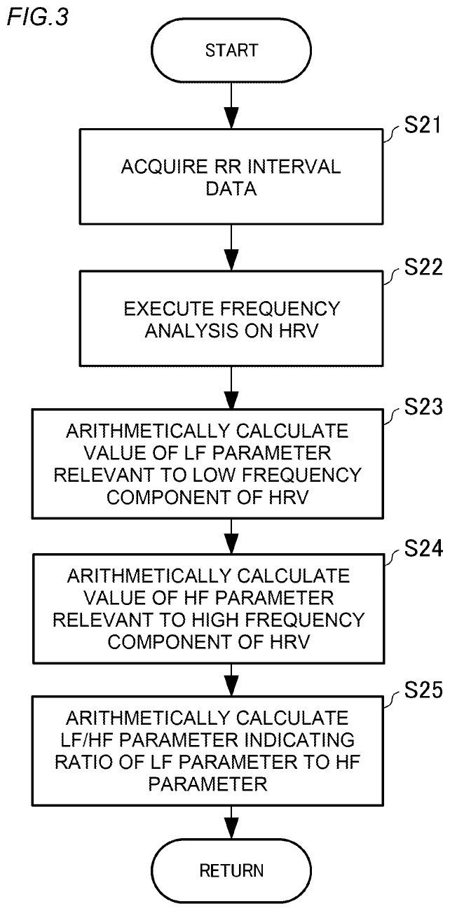

Next, in a step S2, the controller 2 acquires values of parameters (an LF parameter, an HF parameter, and an LF/HF parameter) relevant to an autonomic nerve function of the patient P in every first time interval (e.g. every time unit within 1 to 10 seconds) based on the electrocardiogram data. A process of acquiring the parameters relevant to the autonomic nerve function of the patient P will be described below with reference to FIG. 3. FIG. 3 is a flowchart showing an example of a process of acquiring values of the parameters relevant to the autonomic nerve function of patient P.

As shown in FIG. 3, the controller 2 acquires RR interval data including RR intervals based on the electrocardiogram data in a step S21. Here, each of the RR intervals is a time interval between an R wave of a predetermined heartbeat waveform (QSR waveform) and an R wave of an adjacent heartbeat waveform to the predetermined heartbeat waveform. For example, the controller 2 identifies time instants of peak points of the R waves from the electrocardiogram data. Then, the controller 2 generates RR interval data indicating a change of the RR intervals over time after having identified the RR intervals from the identified time instants of the peak points of the R waves. Next, the controller 2 performs frequency analysis on heart rate variability (HRV) of the patient P (step S22). Specifically, the controller 2 performs frequency analysis (such as wavelet analysis, fast Fourier transform (FFT), or the like) on the RR interval data indicating a variation of the RR intervals over time. Here, the RR interval data may include a plurality of RR intervals Rₙ associated with heartbeat numbers n (n is a natural number). For example, an n-th RR interval Rₙ is a time interval between a time instant of a peak point of an R wave of an n-th heartbeat waveform Wₙ and a time instant of a peak point of an R wave of an (n+1)th heartbeat waveform Wₙ₊₁.

Next, the controller 2 acquires an LF parameter relevant to a low frequency component (LF) of the heart rate variability (HRV) in every first time interval (e.g. every time unit within 1 to 10 seconds) (step S23). For example, the controller 2 may identify, as the LF parameter relevant to the low frequency component (LF) of the heart rate variability, a value of peak intensity or integrated intensity of a power spectrum of the RR interval data in a low frequency band (of, for example, 0.05 Hz to 0.15 Hz). In particular, to acquire the LF parameter at a time instant tₙ, the controller 2 may perform frequency analysis on the RR interval data between a time instant (tₙ−Δt) and the time instant tₙ so as to acquire the LF parameter at the time instant tₙ. Next, to acquire the LF parameter at a time instant tₙ₊₁, the controller 2 may perform frequency analysis on the RR interval data between a time instant (tₙ₊₁−Δt) and the time instant tₙ₊₁ so as to acquire the LF parameter at the time instant tₙ₊₁. Here, tₙ₊₁ is equal to tₙ÷first time interval.

Next, the controller 2 acquires an HF parameter relevant to a high frequency component (HF) of the heart rate variability (HRV) in every first time interval (step S24). For example, the controller 2 may identify, as the HF parameter relevant to the high frequency component (HF) of the heart rate variability, a value of peak intensity or integrated intensity of a power spectrum of the RR interval data in a high frequency band (of, for example, 0.15 Hz to 0.40 Hz). In particular, to acquire the HF parameter at the time instant tₙ, the controller 2 may perform frequency analysis on the RR interval data between the time instant (tₙ−Δt) and the time instant tₙ so as to acquire the HF parameter at the time instant tₙ. Next, to acquire the HF parameter at the time instant tₙ₊₁, the controller 2 may perform frequency analysis on the RR interval data between the time instant (tₙ₊₁−Δt) and the time instant tₙ₊₁ so as to acquire the HF parameter at the time instant tₙ₊₁. The HF parameter is a parameter relevant to a parasympathetic nerve function of the patient P. For example, when the value of the HF parameter is smaller than a threshold during a predetermined period, a medical worker can judge that the parasympathetic nerve function of the patient P has declined.

Next, in a step S25, the controller 2 arithmetically calculates an LF/HF parameter (=LF parameter÷HF parameter) indicating a ratio of the LF parameter to the HF parameter in every first time interval. The LF/HF parameter is a parameter relevant to a sympathetic nerve function of the patient P. When, for example, the value of the LF/HF parameter is smaller than a predetermined threshold during the predetermined period, the medical worker can judge that the sympathetic nerve function of the patient P has declined.

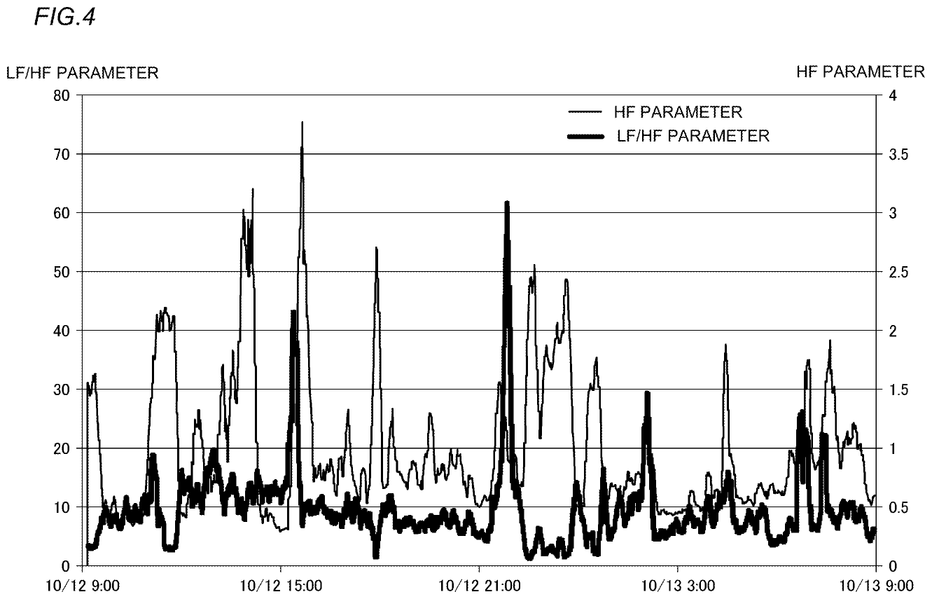

Thus, the controller 2 can acquire the values of the parameters relevant to the autonomic nerve function of the patient P from the electrocardiogram data. In particular, the controller 2 can acquire the value of the LF/HF parameter (an example of a first parameter) relevant to the sympathetic nerve function of the patient P and the value of the HF parameter (an example of a second parameter) relevant to the parasympathetic nerve function of the patient P from the electrocardiogram data. As shown in FIG. 4, the controller 2 can display a trend graph (an example of a first trend graph) indicating a change of the LF/HF parameter over time, and a trend graph (an example of a second trend graph) indicating a change of the HF parameter over time on a display screen of the display 5. After having performed moving average processing on the value of the LF/HF parameter acquired in every first time interval, the controller 2 may display, on the display screen, the trend graph of the value of the LF/HF parameter which has been subjected to the moving average processing. In a similar manner, the controller 2 may display, on the display screen, the trend graph of the value of the HF parameter which has been subjected to the moving average processing.

In addition, the controller 2 may acquire parameters relevant to the autonomic nerve function of the patient P from blood pressure data indicating systolic blood pressure variability over time in place of the electrocardiogram data. In this case, the controller 2 performs frequency analysis (e.g. wavelet analysis, fast Fourier transform (FFT), or the like) on the systolic blood pressure variability (BPV) of the patient P after having acquired the blood pressure data from a not-shown blood pressure sensor. Specifically, the controller 2 acquires a BPV_LF parameter relevant to a low frequency component (LF) of the BPV after having performed frequency analysis on the blood pressure data. The BPV_LF parameter is a parameter relevant to the sympathetic nerve function of the patient P. When, for example, the value of the BPV_LF parameter is smaller than a predetermined threshold during a predetermined period, the medical worker can judge that the sympathetic nerve function of the patient P has declined.

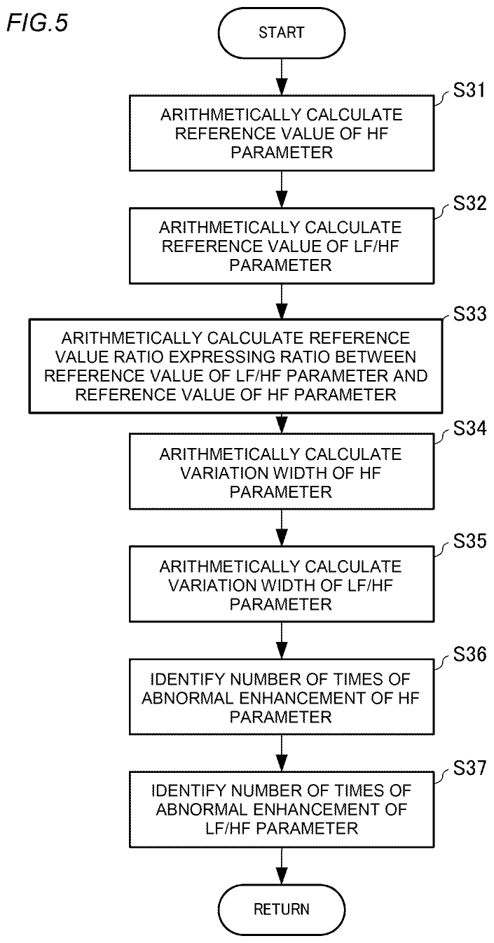

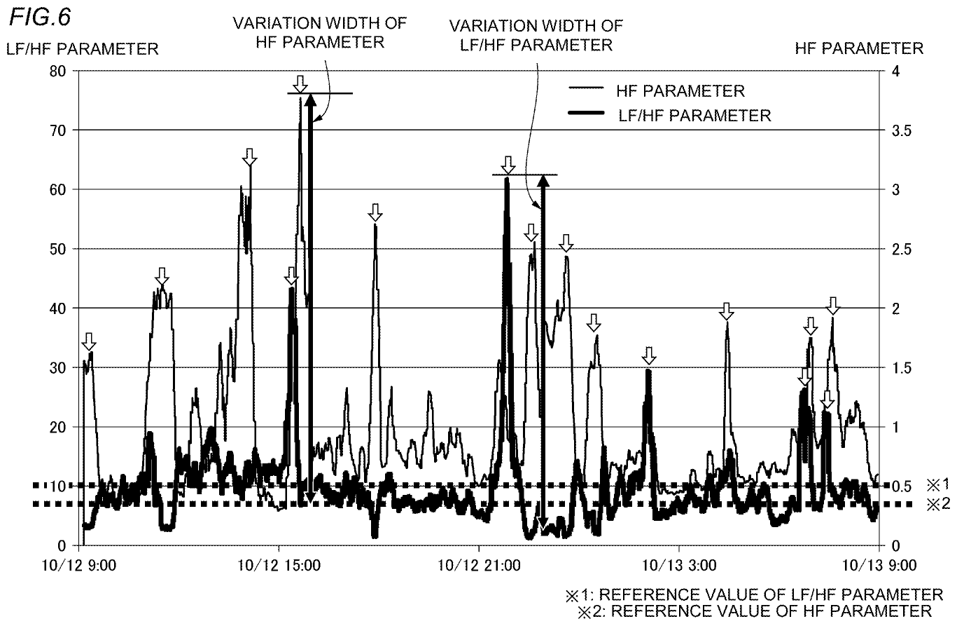

Return now to FIG. 2. The controller 2 acquires abnormality index values indicating an extent of abnormality in the autonomic nerve of the patient P based on the values of the HF parameter and the LF/HF parameter acquired in every first time interval (step S3). A process of acquiring the abnormality index values will be described below with reference to FIG. 5 and FIG. 6. FIG. 5 is a flowchart showing an example of the process of acquiring the abnormality index values. FIG. 6 is a diagram for explaining the abnormality index values. The controller 2 may arithmetically calculate the abnormality index values based on the values of the HF parameter and the LF/HF parameter acquired in every first time interval or may arithmetically calculate the abnormality index values based on the values of the HF parameter and the LF/HF parameter which have been subjected to the moving average processing. Seven abnormality index values will be introduced by way of example in the following description. However, the abnormality index values are not limited thereto.

First, in a step S31, the controller 2 arithmetically calculates a reference value of the HF parameter which is one of the abnormality index values. For example, as shown in FIG. 6, the reference value of the HF parameter is 0.4. Three calculation methods for the reference value of the HF parameter will be described below.

(First Calculation Method for Reference Value of HF Parameter)

First, the controller 2 identifies a median, a mean or a mode of the HF parameter in every second time interval (e.g. every time unit within 1 to 10 minutes) that is wider than the first time interval. For example, after having divided a plurality of values of the HF parameter acquired in every time unit selected from 1 to 10 minutes for 24 hours, the controller 2 arithmetically calculates medians, means or modes of the HF parameter in the divided time units respectively. Next, the controller 2 further arithmetically calculates a median, a mean or a mode of a data group consisting of the medians, the means or the modes of the HF parameter arithmetically calculated in the divided time units respectively. The median, the mean or the mode of the data group arithmetically calculated thus is set as the reference value of the HF parameter. Thus, according to the first calculation method for the reference value, the reference value of the HF parameter prevented from being affected by abnormal enhancement of the HF parameter or abnormal fluctuation caused by noise or the like. The controller 2 specifies a median, a mean, or a mode of the HF parameter in every data interval. The data interval corresponds to the first time interval (e.g. the time unit within 1 to 10 seconds) narrower than the second time interval. Further, the first time interval and the second time interval used in the first calculation method for the reference value of the HF parameter are not limited to the aforementioned time intervals but may be time intervals set desirably by the medical worker or during product manufacturing.

(Second Calculation Method for Reference Value of HF Parameter)

First, the controller 2 identifies a plurality of values of the HF parameter smaller than a threshold (e.g. 1.5) of the HF parameter. Then, the controller 2 calculates, as the reference value of the HF parameter, a median, a mean or a mode of a data group consisting of the plurality of values of the HF parameter smaller than the threshold of the HF parameter. The second calculation method also can calculate the reference value of the HF parameter prevented from being affected by abnormal enhancement of the HF parameter or abnormal fluctuation caused by noise or the like.

(Third Calculation Method for Reference Value of HF Parameter)

The controller 2 identifies a data group consisting of a plurality of values of the HF parameter from which values corresponding to upper N % (0<N<100) of the values of the HF parameter have been excluded. Next, the controller 2 calculates a median, a mean or a mode of the identified data group, as the reference value of the HF parameter. Likewise, the third calculation method can also calculate the reference value of the HF parameter prevented from being affected by abnormal enhancement of the HF parameter or abnormal fluctuation caused by noise or the like.

As shown in FIG. 5, the controller 2 arithmetically calculates a reference value of the LF/HF parameter which is one of the abnormality index values in a step S32. For example, as shown in FIG. 6, the reference value of the LF/HF parameter is 10. The controller 2 can calculate the reference value of the LF/HF parameter by a method similar to or the same as any of the aforementioned three calculation methods for the reference value of the HF parameter.

Next, in a step S33, the controller 2 arithmetically calculates a reference value ratio expressing a ratio between the reference value of the LF/HF parameter and the reference value of the HF parameter as one of the abnormality index values. The reference value ratio may be defined as (reference value of LF/HF parameter)/(reference value of HF parameter) or may be defined as (reference value of HF parameter)/(reference value of LF/HF parameter). When the reference value ratio is defined as (reference value of LF/HF parameter)/(reference value of HF parameter), the value of the reference value of the LF/HF parameter is 10, and the value of the reference value of the HF parameter is 0.4, the value of the reference value ratio is 25.

Next, in a step S34, the controller 2 arithmetically calculates a variation width of the HF parameter which is one of the abnormality index values. As shown in FIG. 6, the variation width of the HF parameter is a difference between a maximum value and a minimum value of the HF parameter. The controller 2 arithmetically calculates the variation width of the HF parameter after having identified the maximum value and the minimum value of the acquired values of the HF parameter.

Next, in a step S35, the controller 2 arithmetically calculates a variation width of the LF/HF parameter which is one of the abnormality index values. As shown in FIG. 6, the variation width of the LF/HF parameter is a difference between a maximum value and a minimum value of the LF/HF parameter. The controller 2 arithmetically calculates the variation width of the LF/HF parameter after having identified the maximum value and the minimum value of the acquired values of the LF/HF parameter.

Next, in a step S36, the controller 2 arithmetically calculates the number of times of abnormal enhancement of the HF parameter which is one of the abnormality index values. The number of times of abnormal enhancement of the HF parameter expresses the number of peak values of the HF parameter exceeding the threshold of the HF parameter. When, for example, the threshold of the HF parameter is set at 1.5, the number of times of abnormal enhancement of the HF parameter is 11 (see the number of arrows added to the trend graph of the HF parameter), as shown in FIG. 6. It can be also said that the number of times of abnormal enhancement of the HF parameter expresses the number of waveforms having the peak values exceeding the threshold in the trend graph of the HF parameter. The threshold of the HF parameter may be allowed to be set suitably by the medical worker.

Next, in a step S37, the controller 2 arithmetically calculates the number of times of abnormal enhancement of the LF/HF parameter which is one of the abnormality index values. The number of times of abnormal enhancement of the LF/HF parameter expresses the number of peak values of the LF/HF parameter exceeding the threshold of the LF/HF parameter. When, for example, the threshold of the LF/HF parameter is set at 20, the number of times of abnormal enhancement of the LF/HF parameter is 5 (see the number of arrows added to the trend graph of the LF/HF parameter), as shown in FIG. 6. It can be also said that the number of times of abnormal enhancement of the LF/HF parameter expresses the number of waveforms having the peak values exceeding the threshold in the trend graph of the LF/HF parameter. The threshold of the LF/HF parameter may be allowed to be set suitably by the medical worker.

In this manner, the seven abnormality index values are calculated through the processings of the steps shown in FIG. 5. The sequence of the processings of the steps shown in FIG. 5 is not limited particularly.

In addition, the calculation methods for the abnormality index values are not limited particularly, and the aforementioned contents thereof are merely exemplified. For example, in the step S34, the variation width of the HF parameter which is one of the abnormality index values arithmetically calculated by the controller 2 may be a difference between the maximum value of the HF parameter and the reference value of the HF parameter.

In the step S35, the variation width of the LF/HF parameter may be a difference between the maximum value of the LF/HF parameter and the reference value of the LF/HF parameter.

In the step S36, the number of times of abnormal enhancement of the HF parameter may be the number of the peak values of the HF parameter exceeding the reference value of the LF/HF parameter.

Further, in the step S36, the number of times of abnormal enhancement of the LF/HF parameter may be the number of the peak values of the LF/HF parameter exceeding the reference value of the LF/HF parameter.

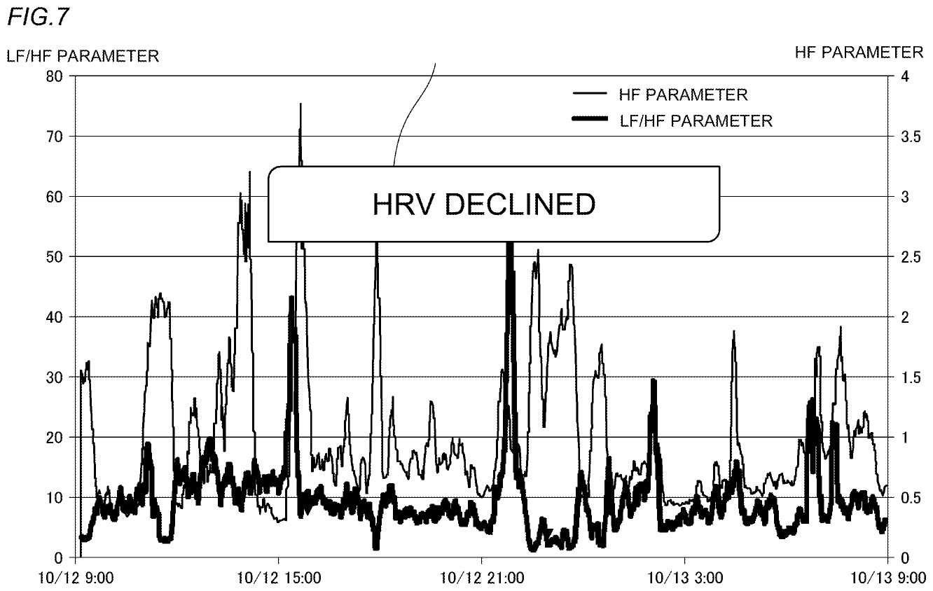

Return now to FIG. 2. In a step S4, the controller 2 displays information relevant to the arithmetically calculated abnormality index values on the display screen of the display 5. After having determined message information M based on the arithmetically calculated abnormality index values (the reference values, the reference value ratio, the variation widths and the numbers of times of abnormal enhancement), the controller 2 may display the message information M in a display area in which the trend graph of the HF parameter and the trend graph of the LF/HF parameter have been simultaneously displayed, for example, as shown in FIG. 7. In this respect, when the reference value of the HF parameter is lower than a normal range, the reference value of the LF/HF parameter is lower than a normal range, the variation width of the HF parameter is lower than a normal range and the variation width of the LF/HF parameter is lower than a normal range, message information M indicating “HRV declined” may be displayed on the display screen. On the other hand, when the number of times of abnormal enhancement of the HF parameter or the number of times of abnormal enhancement of the LF/HF parameter is higher than a normal range, message information M indicating “HRV varied” may be displayed on the display screen.

The normal range of each of the abnormality index values is shown by way of example in the following table.

TABLE 1

| Abnormality Index Value | Normal Range |
|---|---|
| reference value of HF parameter | 1 to 5 |
| reference value of LF/HF parameter | 10 to 50 |
| variation width of HF parameter | 1 to 5 |
| variation width of LF/HF parameter | 10 to 50 |
| number of times of abnormal enhancement of HF | 0 to 10 |
| parameter | |
| number of times of abnormal enhancement of LF/HF | 0 to 10 |
| parameter | |

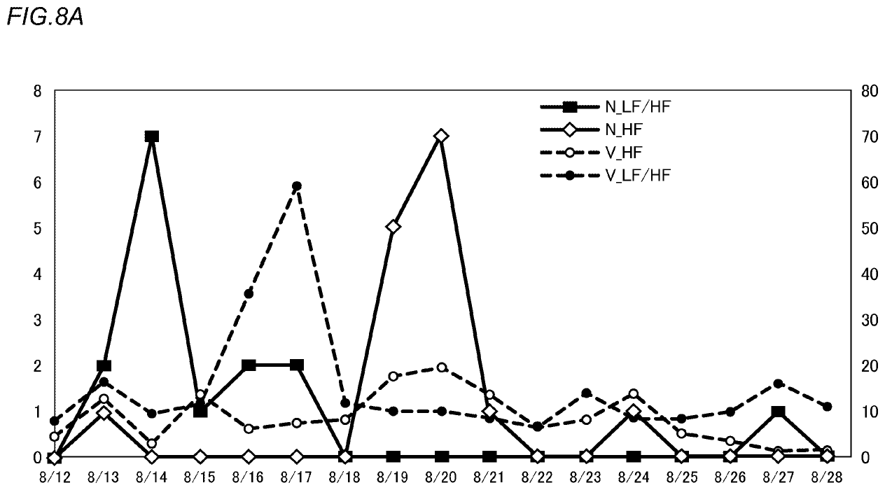

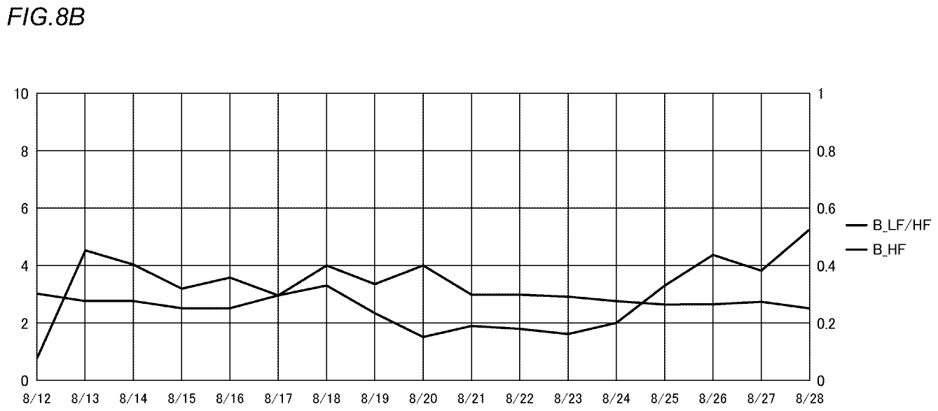

As shown in FIGS. 8A and 8B, the controller 2 may display trend graphs indicating values of the abnormality index values as the information relevant to the abnormality index values on the display screen of the display 5. The following values of the abnormality index values are shown in the trend graphs shown in FIGS. 8A and 8B. In particular, FIG. 8A shows the trend graph of the variation width of the HF parameter, the trend graph of the variation width of the LF/HF parameter, the trend graph of the number of abnormal enhancement of the HF parameter, and the trend graph of the number of abnormal enhancement of the LF/HF parameter, as the information relevant to the abnormality index values. FIG. 8B shows the trend graph of the reference value of the HF parameter and the trend graph of the reference value of the LF/HF parameter as the information relevant to the abnormality index values. In the trend graphs shown in FIGS. 8A and 8B, the scale interval of the abscissa is one day. However, the scale interval of the abscissa may be several days, several hours, several weeks or any time interval.

### (Abnormality Index Values Shown in FIGS. 8A and 8B)

Value of reference value of HF parameter (B_HF)
Value of reference value of LF/HF parameter (B_LF/HF)
Value of variation width of HF parameter (V_HF)
Value of variation width of LF/HF parameter (V_LF/HF)
Value of number of times of abnormal enhancement of HF parameter (N_HF)
Value of number of times of abnormal enhancement of LF/HF parameter (N_LF/HF)

In addition, the normal ranges of the abnormality index values may be visibly displayed in the trend graphs showing the values of the abnormality index values.

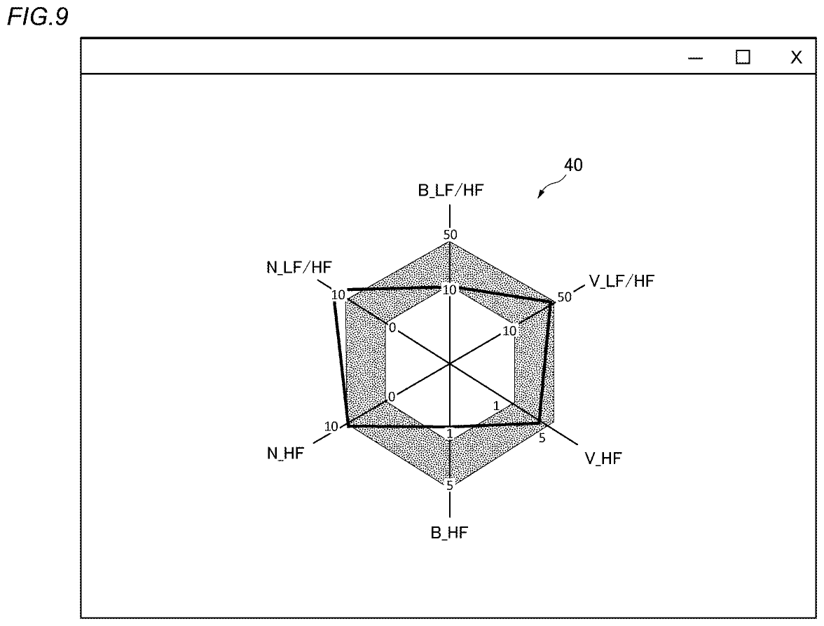

As shown in FIG. 9, the controller 2 may display, on the display screen of the display 5, a radar chart 40 indicating the values of the abnormality index values as the information relevant to the abnormality index values. In addition, in the radar chart 40, the normal ranges of the abnormality index values may be visibly displayed.

According to the present embodiment, the information relevant to the abnormality index values is displayed on the display screen of the display 5. Thus, the medical worker can intuitively grasp the extent of abnormality in the autonomic nerve function of the patient P by visibly recognizing the information relevant to the abnormality index values (the reference values, the reference value ratio, the variation widths, and the numbers of times of abnormal enhancement). In addition, if the medical worker does not have enough experience, the medical worker has difficulty in accurately grasping the extent of the disease of the patient P relevant to the abnormality in the autonomic nerve function by only visibly recognizing the waveforms indicating the changes of the parameters relevant to the autonomic nerve function of the patient P over time.

On the other hand, by use of the processing apparatus 1 according to the present embodiment, even the inexperienced medical worker can estimate an extent of a disease (such as head injury, spinal cord damage, delirium, tetanus, or sepsis) of the patient P relevant to abnormality in his/her autonomic nerve function by visibly recognizing the information relevant to the abnormality index values. For example, by visibly recognizing the radar chart 40 shown in FIG. 9, the medical worker can estimate severity of the tetanus emerging in the patient P. By visibly recognizing the trend graphs shown in FIGS. 8A and 8B, the medical worker can estimate or grasp a prognostic condition of the tetanus emerging in the patient P. Thus, it is possible to provide the processing apparatus 1 that can contribute to diagnosis of the disease of the patient P relevant to the abnormality in his/her autonomic nerve function.

According to the present embodiment, the message information M, the trend graph of the HF parameter, and the trend graph of the LF/HF parameter are simultaneously displayed as the information relevant to the abnormality index values on the display screen. Accordingly, the medical worker can intuitively grasp the extent of the abnormality in the autonomic nerve function of the patient P by visibly recognizing the message information M and the two trend graphs.

In the present embodiment, the message information M and the radar chart 40 are displayed as the information relevant to the abnormality index values on the display screen. However, the information relevant to the abnormality index values is not limited thereto. For example, numerical information about the abnormality index values (the reference values, the reference value ratio, the variation widths and the numbers of times of abnormal enhancement) may be displayed on the display screen together with the two trend graphs. In addition, the value of the reference value ratio may be displayed as one of the abnormality index values in the radar chart 40.

In another embodiment, one or a combination of two or more of the trend graph of the HF parameter, the trend graph of the LF/HF parameter, the message information M, the radar chart 40, the numerical information of the abnormality index values (the reference values, the reference value ratio, the variation widths, and the numbers of times of abnormal enhancement), and the trend graphs showing the values of the abnormality index values may be displayed as the information relevant to the abnormality index values on the display screen.

In addition, in order to implement the processing apparatus 1 according to the present embodiment by software, the physiological information processing program may be incorporated in advance into the storage device 3 or the ROM. Alternatively, the physiological information processing program may be stored in a computer-readable storage medium such as a magnetic disk (such as an HDD or a floppy disk), an optical disk (such as a CD-ROM, a DVD-ROM, or a Blu-ray (registered trademark) disk), a magneto-optical disk (such as an MO), or a flash memory (such as an SD card, a USB memory, or an SSD). In this case, the physiological information processing program stored in the storage medium may be incorporated into the storage device 3. Further, the processor may execute the program loaded on the RAM after the program incorporated into the storage device 3 has been loaded onto the RAM. In this manner, the physiological information processing method according to the present embodiment can be executed by the processing apparatus 1.

In addition, the physiological information processing program may be downloaded from a computer on the communication network through the network interface 4. Also in this case, the downloaded program may be likewise incorporated into the storage device 3.

The embodiment of the present disclosure has been described above. However, the technical scope of the present disclosure should not be limitedly interpreted by the description of the present embodiment. The present embodiment is exemplified. It should be understood by those skilled in the art that various changes can be made on the embodiment within the scope of the disclosure described in CLAIMS. The technical scope of the present disclosure should be determined based on the scope of the disclosure described in CLAIMS and the scopes of equivalents thereto.

1. A physiological information processing apparatus comprising:
a processor, and
a memory that stores a computer-readable command,
wherein when the computer-readable command is executed by the processor, the physiological information processing apparatus is configured to:
acquire physiological information data indicating physiological information of a subject;
acquire a value of a parameter relevant to an autonomic nerve function of the subject in each of first time intervals based on the physiological information data;
acquire an abnormality index value indicating an extent of abnormality in the autonomic nerve function of the subject based on the values of the parameter acquired in the first time intervals, and
display information relevant to the abnormality index value.

2. The physiological information processing apparatus according to claim 1, wherein the parameter includes a first parameter relevant to a sympathetic nerve function of the subject, and a second parameter relevant to a parasympathetic nerve function of the subject.

3. The physiological information processing apparatus according to claim 1, wherein the abnormality index value includes a reference value of the parameter.

4. The physiological information processing apparatus according to claim 2, wherein the abnormality index value includes a first reference value of the first parameter, and a second reference value of the second parameter.

5. The physiological information processing apparatus according to claim 4, wherein the abnormality index value further includes a reference value ratio expressing a ratio between the first reference value and the second reference value.

6. The physiological information processing apparatus according to claim 1, wherein the abnormality index value includes a number of times of abnormal enhancement of the parameter; and the number of times of abnormal enhancement expresses a number of peak values of the parameter exceeding a threshold of the parameter.

7. The physiological information processing apparatus according to claim 1, wherein the abnormality index value includes a variation width of the parameter that is a difference between a maximum value and a minimum value of the parameter.

8. The physiological information processing apparatus according to claim 3,
wherein the physiological information processing apparatus is configured to:
identify a median, a mean or a mode of the parameter in each of second time intervals wider than the first time interval, and
calculate the reference value based on the medians, the means or the modes of the parameter identified in the second time intervals.

9. The physiological information processing apparatus according to claim 3,
wherein the physiological information processing apparatus is configured to:
identify, of the values of the parameter, values of the parameter smaller than a threshold of the parameter, and
calculate, as the reference value of the parameter, a median, a mean or a mode of the values of the parameter smaller than the threshold of the parameter.

10. The physiological information processing apparatus according to claim 3,
wherein the physiological information processing apparatus is configured to calculate, as the reference value of the parameter, a median, a mean or a mode of values of the parameter from which values corresponding to upper N % (0<N<100) of the values of the parameter have been excluded.

11. The physiological information processing apparatus according to claim 1, wherein the physiological information processing apparatus is configured to simultaneously display information relevant to the abnormality index value and a trend graph indicating a change of the parameter over time.

12. The physiological information processing apparatus according to claim 2, wherein the physiological information processing apparatus is configured to simultaneously display information relevant to the abnormality index value, a first trend graph indicating a change of the first parameter over time, and a second trend graph indicating a change of the second parameter over time.

13. The physiological information processing apparatus according to claim 1, wherein the physiological information processing apparatus is configured to display message information determined based on the abnormality index value.

14. The physiological information processing apparatus according to claim 1, wherein the physiological information processing apparatus is configured to display numerical information of the abnormality index value.

15. The physiological information processing apparatus according to claim 1,
wherein the abnormality index value includes a plurality of abnormality index values, and
wherein the physiological information processing apparatus is configured to display a graph indicating the abnormality index values.

16. A physiological information processing method comprising:
a step of acquiring physiological information data indicating physiological information of a subject;
a step of acquiring a value of a parameter relevant to an autonomic nerve function of the subject in each of first time intervals based on the physiological information data;
a step of acquiring an abnormality index value indicating abnormality in the autonomic nerve function of the subject based on the values of the parameter acquired in the first time intervals, and
a step of displaying information relevant to the abnormality index value.

17. A computer-readable storage medium in which a physiological information processing program for making a computer execute a physiological information processing method according to claim 16 is stored.
